(19) United States
(12) Patent Application Publication
(10) Pub. No.: US 2009/0292346 A1
(43) Pub. Date: Nov. 26, 2009

(54) Porous Niobium Oxide as Electrode Material and Manufacturing Process

(75) Inventor: Anna Norlin, Stockholm (SE)

Correspondence Address: SCHIFF HARDIN, LLP;PATENT DEPARTMENT, 233 S. Wacker Drive-Suite 6600, CHICAGO, IL, 60606-6473, US

(73) Assignee: ST. JUDE MEDICAL AB, Jarfalla (SE)

(21) Appl. No.: 11/887,463

(22) PCT Filed: Mar. 31, 2005
(86) PCT No.: PCT/SE2005/000480

§ 371 Date: Sep. 27, 2007

(51) Int. Cl. A61N 1/05; C25D 11/02

(52) U.S. Cl. 607/119; 205/333

(57) ABSTRACT

An implantable medical electrode has an electrically conductive core covered by a stable biocompatible oxide layer. The core contains niobium and the oxide contains a porous niobium oxide. In a process for producing such an implantable electrode, a core of metal or metal alloy containing niobium is connected as an anode in an electrolyte and is subjected to high potential anodic pulses.

### TECHNICAL FIELD OF THE INVENTION

The present application concerns an electrode having as surface a new material and a process for producing this new material. The electrode of the invention is suitable for use in the human or animal body. The new material gives the electrode excellent electrochemical properties.

### BACKGROUND OF THE INVENTION

Individuals suffering from certain heart conditions (e.g. arrhythmia) can restore normal heart rhythm and improve life quality by having a pacemaker implanted. Pacemakers generate electrical impulses that artificially stimulate the heart tissue. The impulses are transferred to the heart by an electrode, often located inside the heart. On the electrode/tissue interface the electrical stimulation current is converted to an ionic current which is able to act in the body. There are three electrochemical mechanisms by which the current can be transferred over the interface: i) non-faradaic charging/discharging of the electrochemical double layer, ii) reversible and iii) irreversible faradaic reactions. The first two mechanisms do not generate by-products and are preferred rather than irreversible reactions, which generate by-products that can be harmful to the body.

To reduce battery drain and increase the lifetime of the pacemaker, the amount of energy delivered by each stimulation pulse should be small. One way to achieve low energy stimulation is to increase the impedance of the electrode by coating it with a material of high dielectric constant. These materials have high ohmic resistance and will inhibit electron transfer through electrochemical reactions, but can transfer stimulation pulses by a capacitive current. In this way, irreversible electrochemical reactions leading to charge loss are avoided, which is also beneficial as there are no net-reactions.

This type of materials has been used as electrode materials in capacitors due to their charge storage properties. For example V. Fischer, H. Stormer, D. Gerthsen, M. Stenzel, H. Zillgen, E. Ivers-Tiffee describe the dielectric properties of a niobium oxide layer in “Niobium as new material for electrolyte capacitors with nanoscale dielectric oxide layers”, Proceedings of the IEEE International Conference on Properties and Applications of Dielectric Materials, v 3 (2003), p 1134-1137. Several attempts have been made to make such electrodes work in reality for pacemaker applications, but without success.

The surface structure is important to implant materials in general and to stimulation electrodes in particular. It well known that surfaces exhibiting specific topography assists the in-growth of tissue and reduces the risk of inflammation. For pacemaker applications, it is also critical that the electrode is capable of delivering sufficient stimulation to ensure activation of the cardiac tissue. Traditionally, the charge delivery area has been increased in order to increase the surface capacitance. However, only a certain enlargement of the electrode area has proven to be useful during the high frequency stimulation processes. This implies certain limitations in traditional electrode design.

So called valve metals, i.e. Ti, Ta, W, Zr, Al, Hf, Nb, form a porous structure when subjected to high anodic potential (over dielectric break-down) pulses in an electrolyte. This process is called plasma electrolytic oxidation, anodic sparc oxidation or micro-arc oxidation (A. L. Yerokhin, X. Nie, A. Leyland, A. Matthews, S. J. Dowey, “Plasma electrolysis for surface engineering” Surface and Coatings Technology, v 122, n 2-3, 15 Dec. 1999, p 73-93) and has been used to produce corrosion and wear resistant surfaces. Recently, it has gained interest within the biomaterial industry due to its bioactive surface structures. The porous oxide formed on Ti exhibits good biocompatibility.

Pt, Ti and TiN coated electrodes are often used in pacemakers. For porous electrodes, only a fraction of the pores is accessible for the electrochemical processes at sufficiently high sweep rates. Owing to the effect of the distributed resistance inside the pores (IR drop), the available capacitance will diminish with increasing sweep rate. This means that, when the sweep rate is increased, the current in the cyclic voltammetry eventually will change character from a near capacitive response to a near resistive response. Rough TiN coated electrodes show charge transfer limitations due to the IR drop. TiN is also very oxidation prone.

Nb₂O₅ is biocompatible and used as implant materials in a variety of applications. Nb₂O is formed naturally on the metal in air. It can be grown by numerous methods including thermal oxidation, passivation in acid and by anodic oxidation. The anodically formed Nb₂O₅-oxide on Nb is an oxygen deficient, highly doped n-type semiconductor with rectifying properties (the current flow in the cathodic direction but not in the anodic) an a bandgap of 3.4 eV.

The object of the invention is to overcome the limitations of presently used implantable electrodes, such as TiN coated electrodes. A further object is to obtain an implantable electrode having a high surface area, a high impedance, a high surface capacitance and good biocompatibility.

An electrode covered with a porous Nb₂O₅-layer has surprisingly proved to be a very efficient electrode.

### SUMMARY OF THE INVENTION

Thus, the invention concerns an electrode having an electrically conductive core covered by a stable biocompatible oxide barrier layer, said core comprising niobium and said oxide comprising porous niobium oxide.

This electrode may be produced according to the invention by connecting a core of metal or metal alloy containing niobium as anode in an electrolyte and submitting it to high potential anodic pulses.

Surprisingly the porous oxide layer obtained on niobium is homogenous on does not show any cracks in spite of the severe treatment. An extensive high potential treatment with anodic pulses have led to flawed oxide layers on other metals (see for example A. Norlin, J. Pan, C. Leygraf, “Investigation of Electrochemical Behavior of Stimulation/Sensing electrode materials—I. Pt, Ti, and TiN-Coated Electrodes” submitted to Journal of Electrochemical Society (2004) and A. Norlin, J. Pan, C. Leygraf, “Investigation of Electrochemical Behavior of Stimulation/Sensing electrode materials—II. Conductive Oxide Coated Electrodes” submitted to Journal of Electrochemical Society (2004)).

The niobium oxide layer obtained by this process is new and has not been described earlier. It shows unexpected high performance when used as electrode layer, especially on a stimulation electrode.

### BRIEF DESCRIPTION OF THE DRAWINGS

FIG. 1 shows a SEM picture of Nb₂O₅ produced by plasma electrolytic oxidation by 125 pulses of 700 V and 10 ms duration.

FIG. 2 shows diagrams over production of porous niobium oxide, a) potential response to applied pulses, b) current response to applied pulses,

FIG. 3 shows SEM pictures of niobium oxide after a) 50 pulses, b) 75 pulses, c) 100 pulses and d) 125 pulses,

FIG. 4 shows a SEM-picture of a cross-section of oxide in back-scatter mode,

FIG. 5 shows Bode plots obtained from impedance spectroscopy of porous and smooth niobium in PBS,

FIG. 6 shows Bode plots obtained from impedance spectroscopy of porous niobium in PBS,

FIG. 7 shows an equivalent circuit used to model the porous Nb₂O₅ electrode and schematic illustration of two-layer oxide on niobium,

FIG. 8 shows Bode plots of porous Nb₂O₅ during anodic polarization,

FIG. 9 shows Bode plots of porous Nb₂O₅ during cathodic polarization,

FIG. 10 is a diagram showing the change in R_{bl} during cathodic polarization

FIG. 11a) is a cyclic voltammogram of porous Nb₂O₅ in PBS at 1, 5, 10, 15 and 20 V/s, and in b) capacitance is plotted vs potential for 1, 5, 10, 15 and 20 V/s,

FIG. 12a) is a diagram showing potential and b) a diagram showing current responses following applied pacemaker pulses of various magnitude,

FIG. 13a) is a diagram showing pulse impedance for various pulse potentials and b) a diagram showing delivered charge,

FIG. 14a and b are EIS spectra showing the sealing process of the pores during a) the first 30 days and b) 30-60 days,

FIG. 15a, b are cyclic voltammograms of porous niobium oxide obtained at 50 mV/s in PBS. a) shows change in current response with number of cycles, b) shows zoom of oxidation peak,

FIG. 16a-f are SEM pictures showing the niobium oxide obtained after 50, 75, 100, 125, 150 and 200 pulses, respectively.

### DETAILED DESCRIPTION OF THE INVENTION

The invention concerns an implantable electrode having an electrically conductive core covered by a stable biocompatible oxide barrier layer, said core comprising niobium and said oxide comprising porous niobium oxide. The oxide layer comprises suitably an inner compact oxide and an outer porous oxide. The porous oxide may have a pore size of 1 to 20 μm, preferably 2 to 15 μm, especially 3 to 10 μm. The thickness of the barrier oxide layer is suitably 0.5 to 15 μm, preferably 1 to 10 μm and especially 2 to 7 μm. The core comprises niobium. It may be an alloy containing niobium or an essentially pure niobium metal. It is also possible to have a core comprising an inner core made of another metal or metal alloy and covered by a layer of niobium or niobium alloy. Preferably at least the outer layer of the core is made of niobium, most preferably essentially pure niobium.

The invention combines the advantages of the homogenous porous structure of plasma electrolytic oxidation formed Nb₂O₅ with its good electrical properties, such as capacitive stimulation properties.

According to the invention the present electrode is produced by connecting a core of metal or metal alloy containing niobium as anode in an electrolyte and subjecting it to high potential anodic pulses. Suitable electrolytes are a phosphate buffered saline solution and a solution of calcium acetate and calcium glycerophosphate. The pulse magnitude is suitably about 100 to 2000 V, preferably 200 to 1000 V and especially 500 to 1000 V. The pulse duration is suitably about 1 to 20 ms, preferably 2 to 15 ms, especially about 7 to 13 ms. The number of pulses should be at least 40, preferably at least 50, and at most 700, preferably at most 500, especially at most 300. Most preferably the number of pulses is in the interval from 100 to 200.

Also implantable electrodes comprising an electrically conductive core covered by a stable biocompatible oxide barrier layer, where said core comprises one of the valve metals Ti, Ta, W, Zr, Al, Hf and said oxide comprises a porous oxide of this metal produced by high anodic pulses show the same advantages as the electrode having a porous niobium oxide surface.

In the following examples the invention is further illustrated.

### EXAMPLES

In the following examples the performance as a biocompatible electrode with capacitive stimulation properties is investigated. Electrochemical impedance spectroscopy (EIS) is used to investigate the electrode/electrolyte interface and its changes during anodic and cathodic polarization. Cyclic voltammetry (CV) is performed at different sweep rates in the potential range from −2 V to 2 V vs. sat. Ag/AgCl, to account for processes occurring during the pacemaker pulse. Also the electrochemical response of the electrode when subjected to pacemaker pulses is evaluated. All electrochemical measurements are performed in a phosphate-buffered solution (PBS), with Na⁺, K⁺, and Cl⁻ contents similar to those of blood. The surface microstructure is characterized by scanning electron microscopy (SEM) and atomic force microscopy (AFM). The surface composition is analyzed by X-ray photoelectron spectroscopy (XPS) and electron dispersive spectroscopy (EDS).

### Experimental

The nano-porous oxide was investigated by EIS, CV, SEM/EDS, AFM and XPS.

### Electrode Materials

Nb—A 99.99% piece of 1 mm thickness (Alfa Aesa) was polished to surface smoothness of 4000 grit and cleaned 5 minutes by a mixture of ethanol, acetone and distilled water (40:40:20) in an ultrasonic bath.

### Electrolyte and Electrochemical Cell

All measurements were performed at room temperature. The electrolyte used was a phosphate-buffered-saline (PBS) solution, adjusted to pH 7.4 with 1 M NaOH. The solution has a similar ion strength as blood, [Na⁺]=0.17 M, [K⁺]=0.01 M and [Cl⁻]=0.15 M. The cell was a standard three-electrode flat cell (EG&G PARC Flat Cell) with a saturated Ag/AgCl electrode as reference electrode and a Pt mesh as counter electrode. The cell was equipped with a stirrer, adjusted at 750 rpm to remove bubbles on the working electrode during CV-measurements.

### Anodic Oxidation

Polished and cleaned niobium foil was anodically oxidized with 45-125 pulses. A device (UHM0013) built by St Jude Medical AB, Sweden, was used to generate and deliver the high potential pulses. Each pulse had a magnitude of 700 V and 10±2 ms duration. The integrated energy of each pulse was 40 J. Four pulses were applied in series, followed by a two minutes rest period before another pulse series was applied. The voltage and current responses of the materials when exposed to the pulses were recorded by a digital oscilloscope (Tektronix TDS 420A).

The UHM0013-device supplies capacitively delivered pulses, i.e., the pulse starts with the maximum voltage and decays with time. The pulses are not of constant potential, which complicates the analysis of the electrochemical processes occurring during pulsing. The potential response of the materials was directly measured between the working electrode and the Ag/AgCl (sat. KCl) reference electrode. To minimize the potential drop due to the electrolyte resistance, the reference electrode was inserted in a Lugging capillary placed close to the material surface. The current response was measured indirectly by recording the potential over a resistor (15Ω) connected in series with the test cell. The current was then calculated using ohms law.

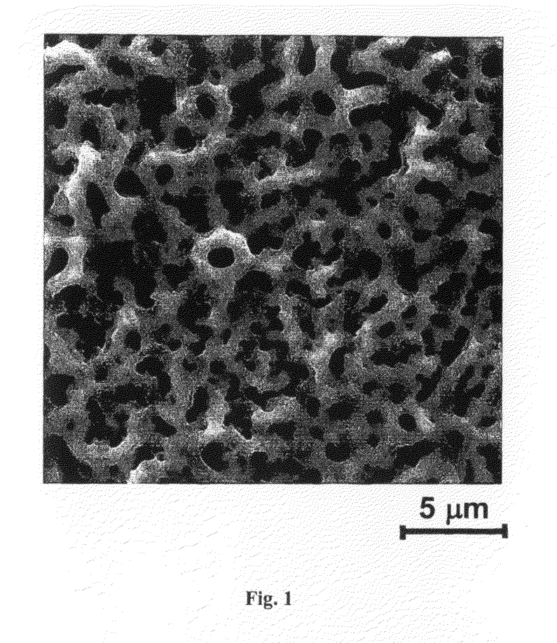

The niobium substrate anodically oxidized by 125 pulses of 700 V and 10 ms duration led to the highly porous Nb₂O₅ shown in the SEM picture in FIG. 1.

### Instruments and Measurements

Electrochemical impedance spectroscopy (EIS)—EIS-measurements of the materials in the solution were performed to characterize the electrode/electrolyte interfaces. EIS-spectra were also obtained for the materials after simulated ageing. All EIS-spectra were collected using an electrochemical interface (Solartron 1287) and a frequency response analyser (Solartron 1250), controlled by a computer with ZPlot software (Scribner Associates, Inc.). The measurements were performed at the open circuit potential (OCP), over a frequency range from 1×10⁴ to 5×10⁻³ Hz. The perturbation amplitude was 10 mV.

Cyclic Voltammetry (CV)—CV was used to study the electrochemical processes that may occur on the electrode surface. The measurements were performed by using either a Solartron 1287, controlled by a computer with CorrWare software (Scribner Associates, Inc.), or an EG&G 273A, controlled by PowerCV software. The CV cycling was performed between −2 V and 2 V vs. Ag/AgCl (sat. KCl). To investigate the influence of the potential sweep rate, the cycling was performed using a range of sweep rates from 50 mV/s to 20 V/s (upper limit of the instrument/software systems).

Pacemaker pulses—Pacemaker pulses were applied between the sample (working electrode) and the counter electrode, using a pulse generator built by St Jude Medical AB, Sweden, which is able to deliver capacitively coupled stimulation and recharging pulses of adjustable potentials and frequencies. The voltage and current responses were recorded by using a digital oscilloscope (Tektronix TDS 420A). The potential response was directly measured between the working electrode and the reference electrode. To minimize the potential drop due to the electrolyte resistance, the reference electrode was inserted in a Lugging capillary and placed close to the sample surface. The responding transient current was measured by recording the potential over a resistor (0.1Ω) connected in series with the test cell (counter electrode). The current was then calculated using ohms law.

Surface analysis—The surface structure of the electrode materials was examined by environmental SEM and AFM. The surface composition of selected samples was analyzed by XPS and EDS.

### Results and Discussion

### Production and Surface Characterization

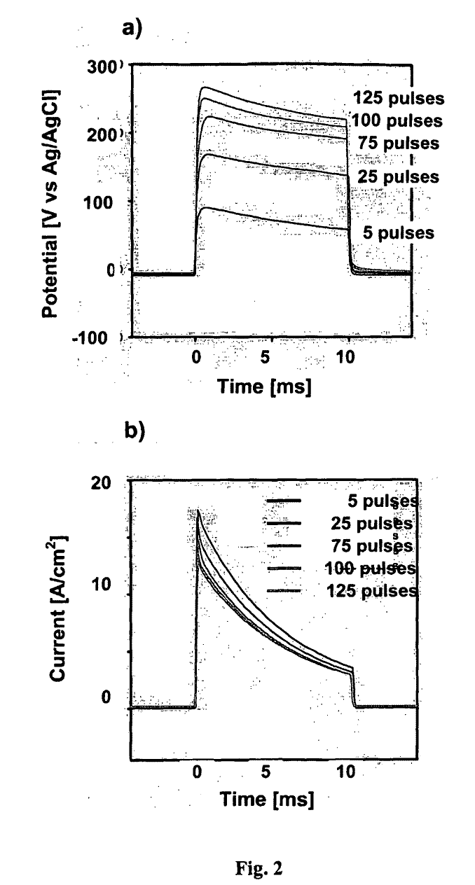

The polished niobium was subjected to high potential pulses of anodical polarity in PBS, according to the method described above. The potential and current responses of the material during pulsing were recorded (FIG. 2a-b). The surface potential increases with consecutive pulses as a consequence of the increasing thickness of the insulating oxide layer. In spite of the increasing surface potential, the current passing the interface decreases with consecutive pulses. Consequently, the charge passed in each pulse is decreased from 85 mC (pulse no 5) to 71 mC (pulse no 125). Calculation of oxide growth, based on 100% conversion factor and an oxide density of 4.36 g/cm³ gives a 6 μm thick oxide layer formed.

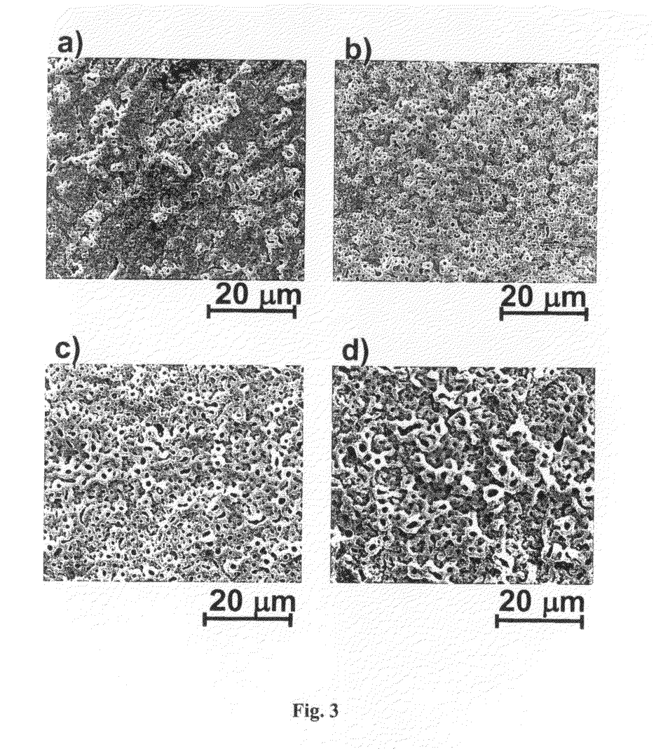

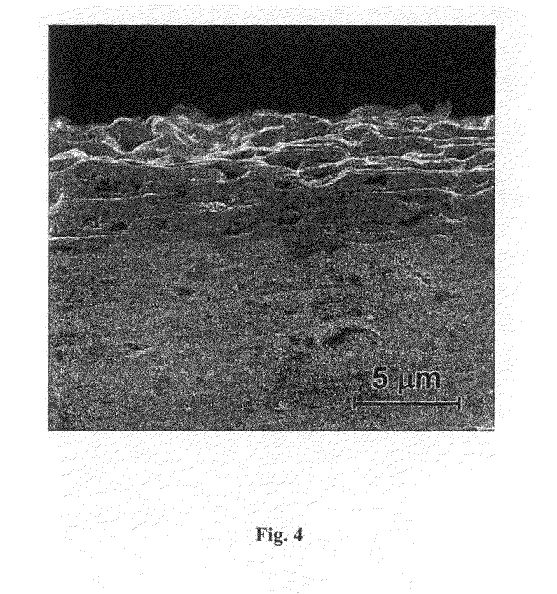

SEM pictures of surface morphology of niobium oxide after 50, 75, 100 and 125 pulses are shown in FIG. 3a-d. After 50 pulses the oxide is covered to approximately 25% by porous oxide. The surface is 100% covered by homogeneous pores of about 1 μm diameter after 75 pulses. Scratch test showed that the oxide adheres well to the substrate and does not flake off close to the scratch. XPS- and XRD-examinations confirm that the oxide is Nb₂O₅ with nano-crystalline structure.

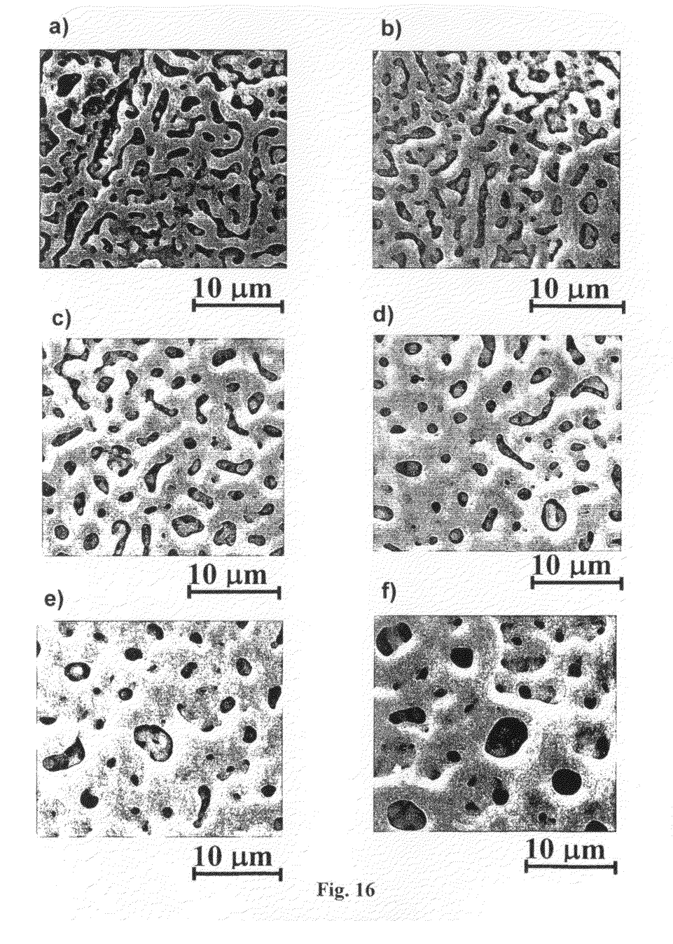

FIGS. 16a-f show another series of SEM-pictures taken after oxidation in PBS with 50, 75, 100, 125, 150 and 200 pulses, respectively, of 700 V. These SEM-pictures show clearly the changes in pore size and number of pores obtained when changing the number of pulses.

A niobium substrate was treated in an electrolyte consisting of a solution of calcium acetate and calcium glycerophosphate with anodic 700 V pulses. In this case P and Ca are incorporated in the oxide as shown I Table 1:

TABLE 1

| Pulses | O | P | Ca | Nb |
|---|---|---|---|---|
| 50 | 71.46 | 2.99 | 1.10 | 24.45 |
| 75 | 71.67 | 3.56 | 1.58 | 23.20 |
| 100 | 71.72 | 4.04 | 2.59 | 21.67 |
| 125 | 72.05 | 4.32 | 3.05 | 20.59 |
| 150 | 71.98 | 4.48 | 3.52 | 20.03 |
| 200 | 72.66* | 4.31* | 3.83* | 19.20* |

The values given are average values, except for the values given for 200 pulses (*) where the analysing apparatus broke down after only one measurement.

The incorporation and concentration of elements such as P and Ca in the oxide may enhance the biocompatibility of the material.

Thus, by changing the number of pulses, the voltage, the electrolyte composition, etc, it is possible to control different oxide and pore parameters as well as thickness of the oxide layer.

Cross-section of the samples show that the porous structure is approximately 2-3 μm thick (FIG. 4). The compact barrier oxide is visible in the mixed SEM and backscatter picture where high atomic elements are shown as lighter gray shade than low atomic elements. Mapping of the chemical composition shows high oxygen content at about 5 μm down into the oxide, indicating a more dense oxygen layer.

### EIS Characterization of the Porous Oxide

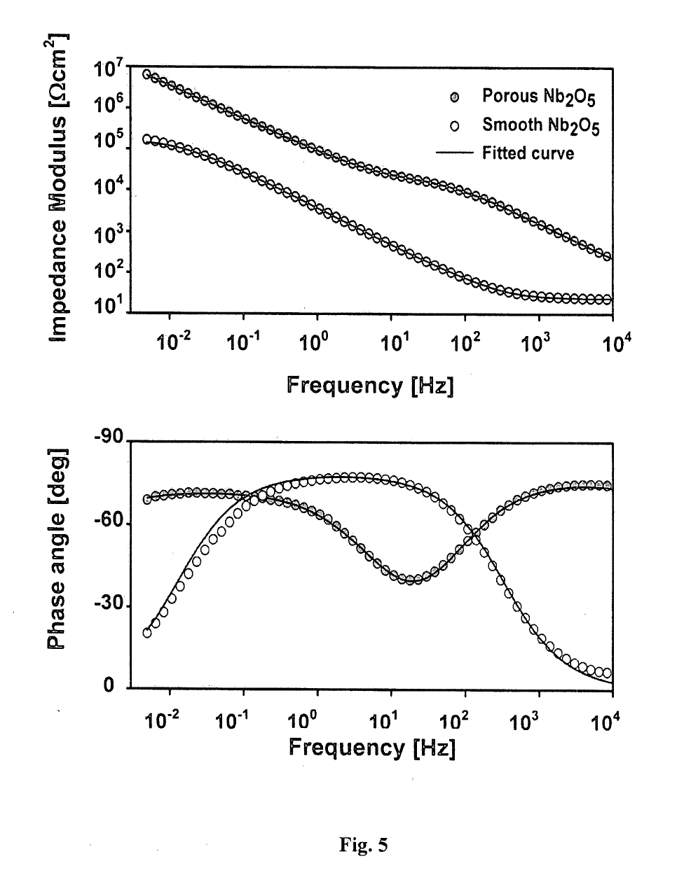

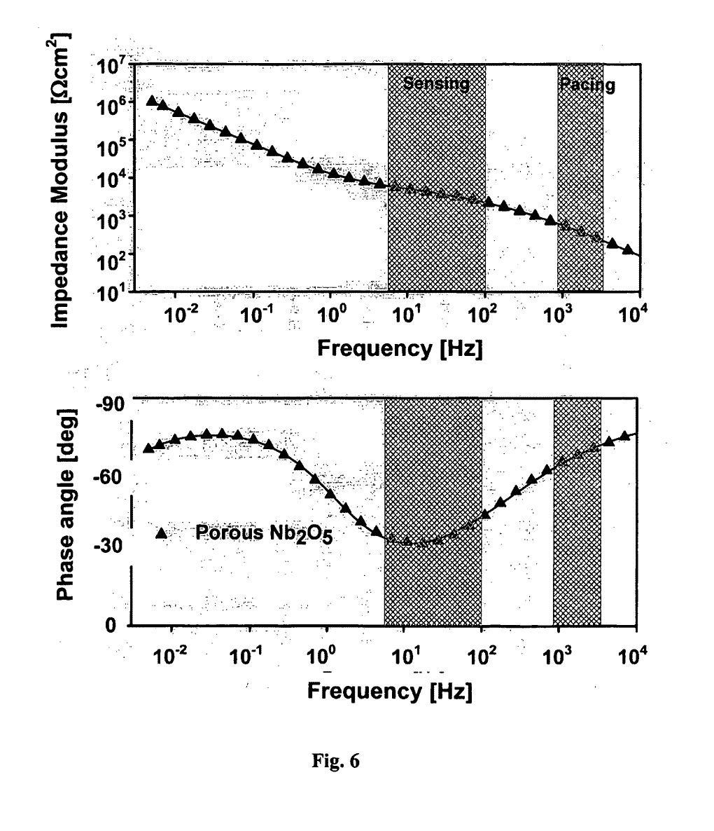

Investigation of the interface by EIS gives valuable information of the electrochemical behaviour of the material over a large frequency range. Only the EIS of niobium oxide after 125 pulses was investigated (100% covered surface). In FIG. 5, the impedance response of smooth untreated niobium is compared to that of porous niobium oxide (125 pulses). The impedance spectra of the untreated smooth niobium electrode show characteristics of a high dielectric material, with high impedance at low frequencies implying a passive oxide on the electrode. The impedance modulus of the porous niobium is significantly higher as a result of the thicker oxide layer. The impedance spectrum of the porous niobium shows the behaviour of two time-constants, as evident by the two distinct peaks in phase angle. This confirms that the oxide film is a two-layer oxide, consisting of an inner compact barrier oxide and an outer porous oxide. This structure was confirmed by SEM in backscatter mode.

Stimulation and sensing processes take place at different frequencies. EIS-investigation can give useful information for designing pacemaker electrodes. FIG. 6 shows impedance spectra in Bode format obtained for the porous Nb₂O₅, with the stimulation and sensing frequency ranges marked in grey.

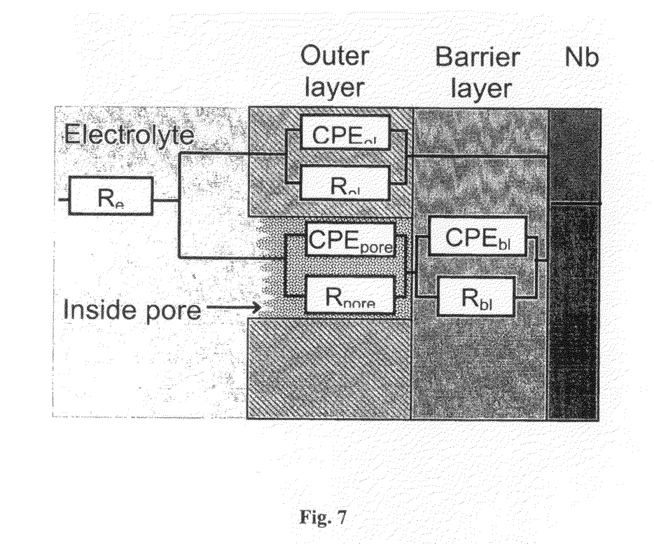

The choice of equivalent circuit is a two-layer model of an oxide film and has been applied in previous studies of oxide films on valve metals. In previous studies the impedance response from the pores was not separated from that of the outer oxide layer. However, to be able to identify the change in impedance response during anodic and cathodic polarization, and to be able to accurately analyze the impedance at intermediate frequencies, the more complex equivalent circuit in FIG. 7 is used here. It is made up of six electric components where Rₑ is the resistance of the electrolyte and CPEₒₗ, and CPE_{bl} are the capacitances of the porous outer oxide layer and the barrier layer, respectively, and R_{bl} is the resistances of the barrier layer. The resistance of the porous oxide layer is very high and could be omitted from the circuit without impairing the quality of the fitted spectra. These components satisfactorily describe the high and low frequency regions of the spectra, but the fit at intermediate frequencies is not very accurate. When a CPE in parallel with a resistor, CPEₚₒᵣₑ and Rₚₒᵣₑ, was introduced into the circuit to account for the frequency dispersion within the pores a satisfactory fit was obtained over the total frequency range.

To account for non-ideal behaviour, the capacitance is represented by a constant phase element (CPE). The impedance of a CPE element is described by:

$${Z}_{CPE} = \frac{1}{{Q ( i ω )}^{η}} ; \qquad ( 1 )$$

where i is the imaginary unit, ω the angular frequency, Q is a constant and η is a mathematic expression (0≦η≦1). For an ideal capacitor, η=1 and Q is the capacitance. The origin of CPE is due to geometric factors such as the roughness and porosity of the electrode as well as surface processes such as adsorption, surface reconstruction and diffusion.

By fitting the model to the experimental data, numeric values of the circuit components are obtained. In the high and low frequency region the impedance response is dominated by CPEₒₗ and CPE_{bl}, respectively. The ranges of numeric values from the experiments are given in Table 2.

TABLE 2

| CPEₒₗ | | CPEₚₒᵣₑ | | CPE_{bl} | | Rₚₒᵣₑ | R_{bl} |
|---|---|---|---|---|---|---|---|
| μF | ηₒₗ | μF | ηₚₒᵣₑ | μF | η_{bl} | kΩ · cm² | MΩ · cm² |
| 0.16-0.17 | 0.98-0.99 | 5-11 | 0.50-0.55 | 20-24 | 0.86-0.90 | 7-8 | 4-6 |

At OCP, the capacitance of the inner barrier oxide layer is 20-24 μF/cm². Assuming a dielectric constant of 42, the thickness of the barrier layer oxide is 1.6-1.9 nm, calculated by;

$$C = {ɛɛ}_{0} \frac{A}{d} \qquad ( 2 )$$

where ∈₀ is the permittivity of vacuum (8.85419×10⁻¹⁴ F/cm), A the area, and C the capacitance of the electrode. The polarization resistance of the barrier layer is high, around 5 MΩcm², indicating a high corrosion resistance, ie, a low rate of niobium release and oxide growth (J. Pan, D. Thierry, C. Leygraf, “Electrochemical impedance spectroscopy study of the passive oxide film in titanium for implant applications”, Electrochim. Acta, 41, 1143 (1996)). The capacitance of the porous outer layer is low, 0.16-0.17 μF/cm² and the resistance of the electrolyte in the pore is in the range of 6-7 kΩcm². The best fit was obtained with ηₚₒᵣₑ close to 0.5, which corresponds to a distributed RC transmission line model (network of distributed resistors and capacitors) which emphasizes the influence of diffusion processes inside the pores.

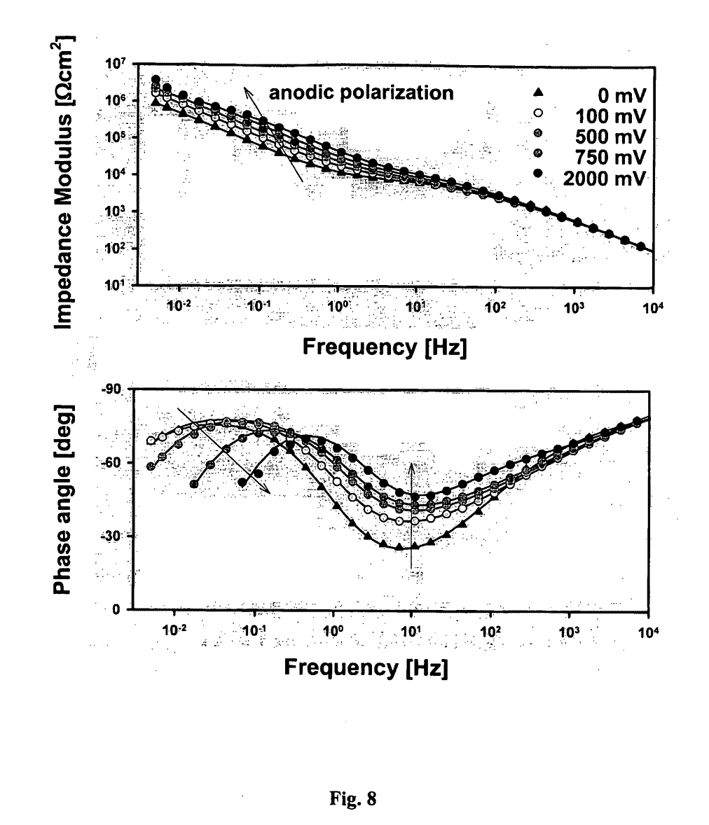

The impedance response during anodic polarization is shown in Bode format in FIG. 8. At high frequencies the impedance modulus and phase angle is relatively unchanged for all magnitudes of polarizations. At low frequencies the impedance modulus increases, indicating some changing properties of the barrier layer.

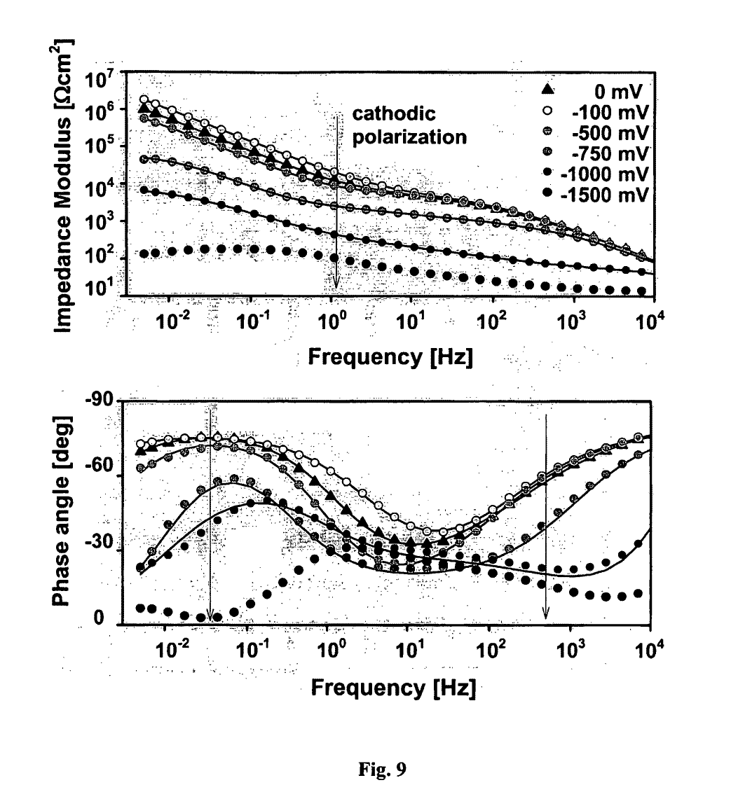

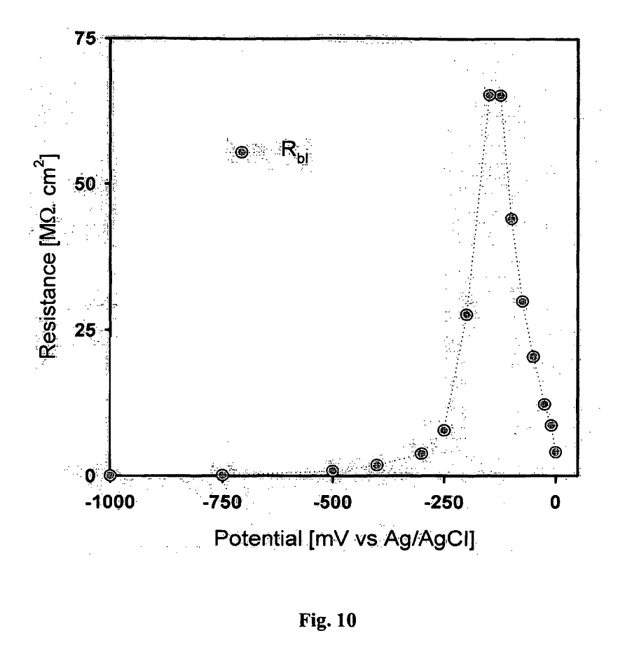

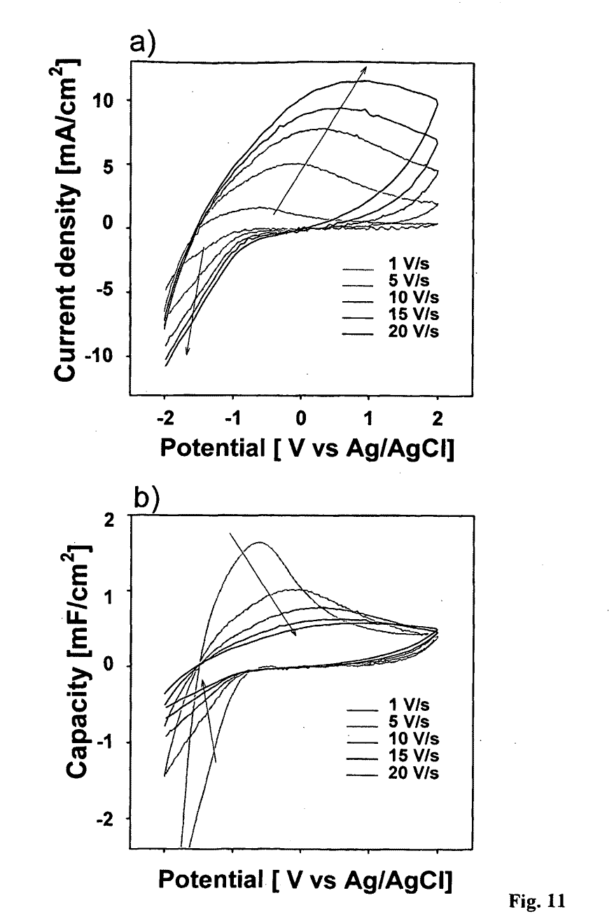

During cathodic polarization the impedance response and the phase angle decreases for both high and low frequencies, as shown in FIG. 9. The resistivity of the barrier layer, R_{bl}, is at its maximum for −150 mV (FIG. 10), then decreases drastically for more cathodic potentials. The resistance within the pores initially increases when the potential become more cathodic until −250 mV, when it decreases again. The capacitance of the barrier layer increases with increasing cathodic polarization and at −750 mV it increases drastically, which might be a result of intercalation of hydrogen into the oxide.

### Aging in PBS Solution

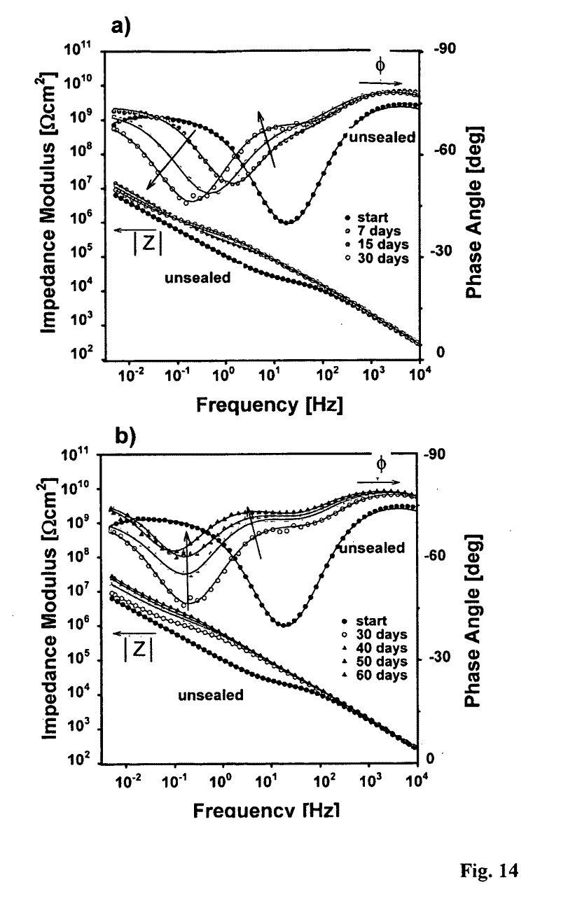

For the newly formed anodic oxide film, the pores are open and filled mainly with electrolyte, which is indicated by a low resistance of the precipitate, Rₚₒᵣₑ. The porous Nb oxide electrode was immersed in PBS for an extended period of time, and its change was monitored by EIS measurements. As shown in FIG. 14, a third peak in phase angle at medium frequencies developed with time. Such change in interfacial characteristics is commonly explained by precipitates filling the pores with time, a process known as pore sealing that occurs on anodic oxide films on valve metals. In this case, the porous part of the oxide film is gradually sealed, and the circuit elements representing the precipitates in the pores changes character.

The variation in the impedance response due to the aging of the porous Nb oxide in PBS provides detailed information about evolution of the oxide film. After 30 days, the impedance modulus at low frequencies also rises (FIG. 14b) while CPE_{bl} decreases, suggesting some thickening of the barrier oxide layer. CPEₒₗ remain essentially unchanged through the exposure time. These results imply that the anodic Nb oxide layer is stable, and precipitates can fill the pores, leading to an increased corrosion resistance.

### Cyclic Voltammetry

CV and capacitance curves (FIGS. 11a-b) of the porous Nb₂O₅ electrodes in the electrolyte show charging/discharging processes and the influence of the sweep rate. To reduce influences from changing film properties on consecutive scans, the film was pretreated by cycling 25 times at 20 V/s. For low sweep rates, very low currents pass the interface at anodic potentials and the current diminishes with increasing over-potential, indicating passive behaviour. For electrode potentials more cathodic than −0.7 V relatively large cathodic current flows, owing to a hydrogen evolution reaction. A small oxidation peak is visible around −1 V in the anodic scan direction, which may be attributed to oxidation of intercalated hydrogen. For increasing scan rates, the anodic oxidation peak becomes broader and more ill-defined and moves towards more anodic potentials for higher sweep rates. For sweep rates lower than 5 V the peak current is not linearly dependent on the sweep rate but for higher sweep rates a linearly dependence was found.

At high sweep-rates, the microstructure of the electrode plays an important role in the current response. When the sweep rate increases, the interface charging or discharging current will increase according to

i_{cap}=C_{interface}s (3)

where the C_{interface} is the combined double layer capacitance and the psuedo-capacitance of the surface bound redox-sites, and s is the sweep-rate. For porous electrodes, only a fraction of the pores is accessible for the electrochemical processes at sufficiently high sweep rates. Owing to the effect of the distributed resistance inside the pores (IR drop), the available capacitance will diminish with increasing sweep rate. This means that, when the sweep rate is increased, the current in the CV eventually will change character from a near capacitive response to a near resistive response.

Despite of its porous nature the Nb₂O₅ electrodes do not fully experience the effect of iR-drop limitations in charge/discharge transfer, for scan rates up to 20 V/s. Even at high scan rates, capacitive features are apparent in the CV. This is exemplified by the increasing current density with increasing scan rates, as well as the typical square like shape of the curves over certain potential ranges. Even though the capacitance diminishes with increasing scan rate, the decrease is not as pronounced as for porous electrodes relying only on charging/discharging of the electrochemical double layer for reversible charge transfer. This indicates that certain psuedo-capacitive redox reactions might contribute to the total charge transfer for Nb₂O₅ electrodes.

Thus, the porous Nb₂O₅ electrodes can utilise its capacitance more effectively at high discharge rates than rough TiN and porous carbon electrodes, which utilise about 5-10% of the capacitance measured by EIS.

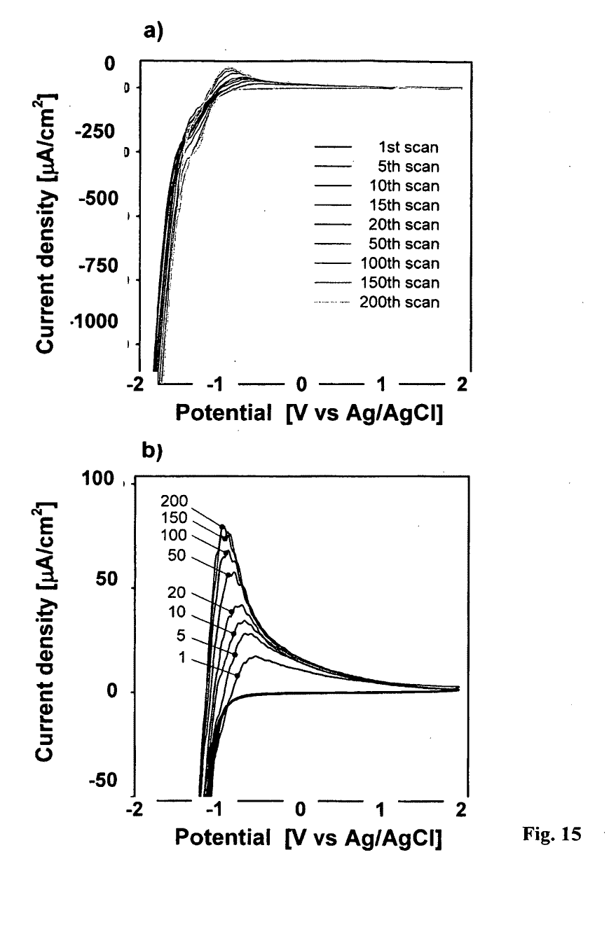

The CV of the porous Nb oxide electrode shows “rectifying” characteristics, FIG. 15a. At anodic potentials the current is very low and diminishes with increasing over-potential. At cathodic potentials below −1 V vs. Ag/AgCl, a distinct increase in current density appear, which can be explained by incorporation of H into the oxide and hydrogen evolution.

In the anodic sweep direction, an oxidation peak appears at about −0.6 V, which is due to oxidation of the H incorporated into the Nb oxide at the cathodic bias, a process known as hydrogen intercalation:

xH⁺+xe⁻+NbO_{2.5}→HₓNbO_{2.5} (5)

The current peak in the anodic sweep increases rapidly for the first 50 consecutive cycles (FIG. 15b), and then reaches a near constant level after 200 cycles. Meanwhile the oxidation peak is shifted to more cathodic potentials with increasing cycles indicating more easily oxidized species within the Nb oxide.

In the cathodic sweep direction, no corresponding reduction peak is observed, but the cathodic current increases significantly around −1 V, followed by a further pronounced increase around −1.6 V. It is suggested to arise from H₂ evolution due to limitations of H incorporation (mass-transport), when HₓNbO_{2.5} is formed on the surface. This also has been attributed to the increase in conductivity of the Nb oxide due to H intercalation. The H acts as a donor impurity, which increases the electronic conductivity of the Nb oxide.

After 200 cycles, the impedance spectrum completely changed character due to H intercalation into the Nb oxide. As H is incorporated, the oxide becomes more conductive, leading to decreased resistance and increased capacitance of the oxide layers.

### Pacemaker Characteristics

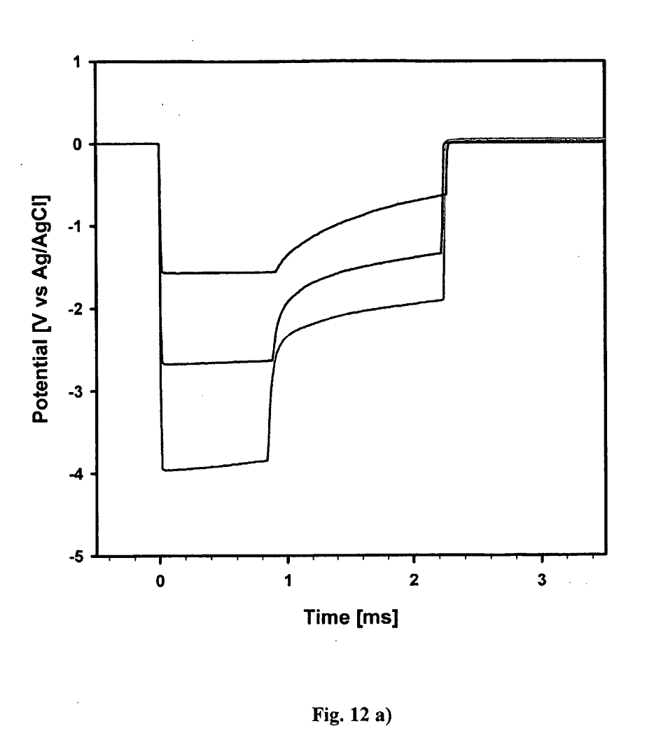

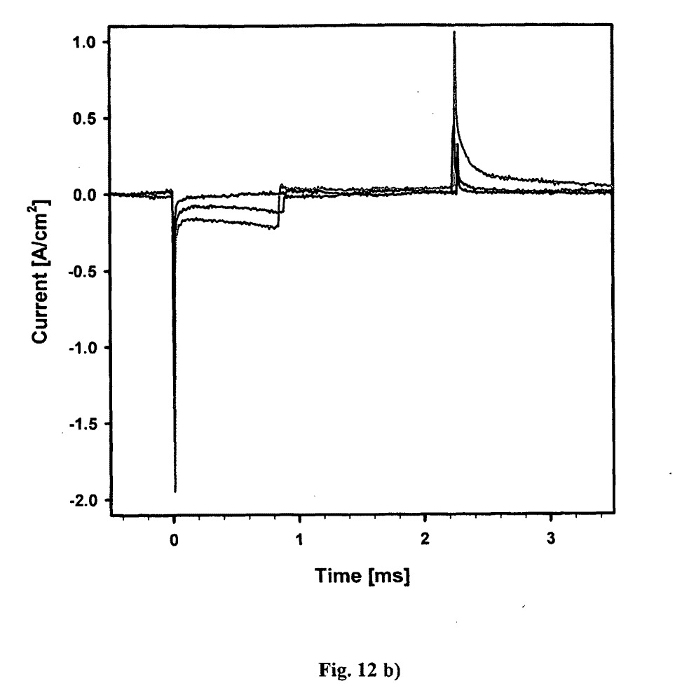

The out-put voltage of the pulses were set to −5, −7.5 and −10 V. Due to the potential drop caused by the electrolyte and the pores, the actual over-potential at the electrode was approximately −0.8, −1.6 and −2.7 V, respectively (relative to the open-circuit potential). The potential and current responses together with the recharging pulse following applied pulses of various magnitude are shown in FIGS. 12a-b. The general shape of the curves is different from those reported previously for smooth and nano-porous carbon.

Pacing polarization—The electrode over-potential decreases only slightly with time during the pacemaker pulse even though the pulse is capacitively delivered. FIG. 9a, shows that the electrode over-potential decays insignificantly over the pulse duration for −0.80 and −1.6 V pulses while for the −2.6 and −4.0 V pulse potentials, the decay is −40 and −100 mV, respectively. Thus, little or no charge is transferred over the interface by faradaic reactions for the lower pulse potentials.

Pacing current and impedance—The current response is shown in FIG. 12b. For −0.8 and −1.7 V potential pulses, the current is delivered in a peak at the first 0.1 ms of the pulse. The exponential decay of the current peak suggests that it originate from pure capacitative charge/discharging of the electrochemical double layer. For the higher potential pulses the peak is followed by a small (100 and 200 mA), almost constant current, that proceeds for as long as the out-put potential is applied. The magnitude of the small current increases with pulse time, implying a decrease of the oxide impedance as the pulse proceeds.

The pacing impedance of the electrode during the pulse can be calculated from the measured potential and current according to:

$$Z ( t ) = \frac{U ( t )}{I ( t )} \qquad ( 4 )$$

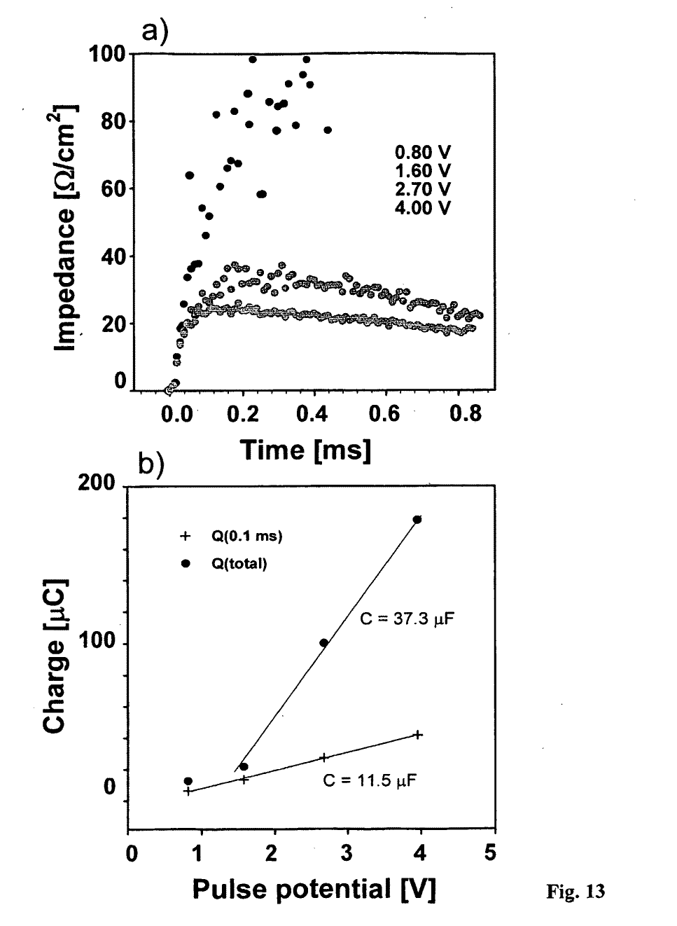

In FIG. 13a the pacing impedance is plotted vs. time during the pulse for different pulse potentials. For the first 0.1 ms, the impedance increases slightly for all pulse potentials. As the pulse proceeds the impedance of the −0.8 and −1.6 V pulses increase drastically while the impedance of the higher potential pulses reach a maximum and then decreases. The high impedance obtained for low pulse potentials can be explained by the lack of faradaic reactions. When the short current peak attributed to charging/discharging of the electrochemical double layer diminish, there is no current flowing across the interface, leading to an extremely high impedance according to equation 4. For higher pulse potentials, there are faradaic currents flowing throughout the entire pulse duration, as seen in FIG. 12b.

If the cathodic current peaks at first 0.1 ms of the pulse are interpreted as originating from the charging/recharging of the electrochemical double layer, the capacitance of the electrode during pulsing can be obtained. In FIG. 13b the charge delivered by the pulse versus the pulse potentials is plotted for both the stimulation pulse and the recharging pulse. The capacitance is obtained from the slope of the plot, and was 12 μF/cm².

After polarization—The trailing edge voltage is the resulting iR-drop following the interruption of current passing the interface, which is determined by the resistance of the electrolyte and the current density. After the applied potential is released, the electrode interface commences to return to equilibrium with the electrolyte, i.e., relaxation process. The after-polarization (relaxation process) is dependent on the over-potential at the trailing edge and interfacial characteristics of the electrode.

Recharging pulse—The application of the recharging pulse does not polarise the electrode to an anodic potential, but merely brings it back to the original potential. The current response of the recharging pulse is clearly visible in FIG. 12b. The origin of anodic current is the oxidation of hydrogen incorporated into the oxide.

### CONCLUSIONS

The electrochemical properties of porous Nb₂O₅ electrodes, produced by plasma electrolytic oxidation, have been investigated in phosphate buffered saline solution. The oxide consists of two layers, one inner thin compact oxide (1.6-1.9 nm) and one thicker porous outer oxide. The interfacial electrochemical behaviour of the porous Nb₂O₅ is dependent on the magnitude and polarization of DC-bias applied to the electrode. At anodic polarization the capacitance of the inner barrier oxide layer decreases due to the increasing thickness of the layer. This leads to increased impedance of the oxide which shows passive behaviour during anodic polarization. At cathodic polarization the oxide changes properties due to intercalation of hydrogen, becomes more conductive and allows cathodic currents to flow. The charging/discharging mechanism remains mainly of capacitive character when the charge/discharge rate in increased. Porous Nb₂O₅ electrodes have extremely high interfacial impedance, and hence low energy loss, when transferring pacemaker pulses.

1.-19. (canceled)

20. An implantable medical electrode comprising:
an electrically conductive core comprising niobium; and
a stable biocompatible oxide barrier layer covering said electrically conductive core, said oxide comprising porous niobium oxide.

21. An implantable medical electrode as claimed in claim 19 wherein said oxide layer comprises an inner compact oxide and an outer porous oxide.

22. An implantable medical electrode as claimed in claim 21 wherein said porous oxide has a pore size in a range between 1 and 20 μm.

23. An implantable medical electrode as claimed in claim 21 wherein said porous oxide has a pore size in a range between 2 and 15 μm.

24. An implantable medical electrode as claimed in claim 21 wherein said porous oxide has a pore size in a range between 3 and 10 μm.

25. An implantable medical electrode as claimed in claim 21 wherein said compact oxide layer has a thickness in a range between 0.5 and 15 μm.

26. An implantable medical electrode as claimed in claim 21 wherein said compact oxide layer has a thickness in a range between 1 and 10 μm.

27. An implantable medical electrode as claimed in claim 21 wherein said compact oxide layer has a thickness in a range between 2 and 7 μm.

28. An implantable medical electrode as claimed in claim 20 wherein said oxide is niobium pentoxide.

29. An implantable medical electrode as claimed in claim 20 wherein said core comprises a niobium layer.

30. An implantable medical electrode as claimed in claim 20 wherein said core is comprised substantially only of niobium.

31. An implantable medical electrode as claimed in claim 20 wherein said oxide is produced by subjecting said core to high potential anodic pulses.

32. An implantable medical electrode as claimed in claim 20 having a configuration forming a pacemaker electrode.

33. An implantable medical electrode as claimed in claim 20 having a configuration forming a defibrillator electrode.

34. A process for producing an implantable medical electrode comprising the steps of:
connecting a core of a metal or metal alloy containing niobium as an anode in an electrical circuit;
placing said core connected as an anode in an electrolyte and subjecting said core to high potential anodic pulses to produce a stable porous and biocompatible niobium oxide layer on said core.

35. A process as claimed in claim 34 comprising using a core comprised of substantially pure niobium.

36. A process as claimed in claim 34 comprising using a phosphate buffered with saline solution as said electrolyte.

37. A process as claimed in claim 34 comprising employing a solution of calcium acetate and calcium glycerophosphate as said electrolyte.

38. A process as claimed in claim 34 comprising employing a pulse magnitude for said high potential anodic pulses in a range between 100 and 2000 volts.

39. A process as claimed in claim 34 comprising employing a pulse magnitude for said high potential anodic pulses in a range between 200 and 1000 volts.

40. A process as claimed in claim 34 comprising employing a pulse magnitude for said high potential anodic pulses in a range between 500 and 1000 volts.

41. A process as claimed in claim 34 comprising employing a pulse duration for said high potential anodic pulses in a range between 1 to 20 ms.

42. A process as claimed in claim 34 comprising employing a pulse duration for said high potential anodic pulses in a range between 2 to 15 ms.

43. A process as claimed in claim 34 comprising employing a pulse duration for said high potential anodic pulses in a range between 7 to 13 ms.

44. A process as claimed in claim 34 comprising employing a number of said high potential anodic pulses in a range between 40 and 700.
